(19) United States
(12) Patent Application Publication
(10) Pub. No.: US 2004/0254404 A1
(43) Pub. Date: Dec. 16, 2004

(54) Method for production of polyolalkyl ethers

(76) Inventors: Thomas Albers, Duesseldorf (DE); Karl Heinz schmid, Mettmann (DE); Ansgar Behler, Bottrop (DE)

Correspondence Address: COGNIS CORPORATION, PATENT DEPARTMENT, 300 BROOKSIDE AVENUE, AMBLER, PA, 19002, US

(21) Appl. No.: 10/494,913

(22) PCT Filed: Oct. 31, 2002
(86) PCT No.: PCT/EP02/12145

(30) Foreign Application Priority Data
Nov. 9, 2001 (DE) 101 55 058.8

(51) Int. Cl. C07C 41/03

(52) U.S. Cl. 568/679

(57) ABSTRACT

A process for making polyol alkyl ether involving: (a) providing a polyol; (b) deprotonating the polyol with a base to form a first reaction product; (c) continuously removing water from the first reaction product to form a second reaction product; (d) providing an alk(en)yl (ether) sulfate; (e) reacting the alk(en)yl (ether) sulfate with the second reaction product to form a third reaction product containing a sulfate salt; (f) precipitating the sulfate salt from the third reaction product by adding from about 10 to 20 mol of water per mol of alk(en)yl (ether) sulfate to the third reaction product, at a temperature of from about 50 to 100° C.; (g) forming an aqueous and/or solid phase containing the polyol alkyl ether; and (h) separating the polyol alkyl ether from the aqueous and/or solid phase.

### FIELD OF THE INVENTION

This invention relates to a new process for the production of polyol alkylethers.

### PRIOR ART

Polyol alkylethers, more particularly alkyl glycerol ethers, are normally produced by deprotonating polyols with strong bases and then reacting the deprotonated product with (alkyl)ether sulfate powders or pastes in the presence of organic solvents. Under these reaction conditions, the aqueous phase accumulating during the reaction through washing of the reaction mixture contains a high percentage of sulfates besides unreacted polyols, so that problems arise during subsequent processing. For this reason, the unreacted polyols present in the aqueous phase cannot be returned to the process. In addition, the disposal as waste of the aqueous phase, which contains relatively large amounts of the polyol, involves significant pollution of the wastewater by sulfates. Where alkyl(ether) sulfate pastes are used, foaming-related problems arise during removal of the water, in addition to which the alkyl(ether) sulfates in the pastes have a tendency to undergo hydrolysis to a fairly significant extent.

Polyol monoalkylethers can also be synthesized by reaction of alkyl halides with alcoholates in the presence of strong bases. A conventional method for the production of alkyl glycerol ethers is synthesis from epichlorohydrin and fatty alcohols via alkyl glycidol ethers and subsequent hydrolytic opening of the epoxide ring. The main disadvantage of these two methods lies in the presence of organochlorinated compounds in the alkyl glycerol ether which cannot therefore be used in cosmetic products in particular.

Accordingly, the problem addressed by the present invention was to provide a process for the production of polyol alkylethers in which the unreacted polyols in the aqueous phase accumulating could be worked up and, hence, further processed by virtue of the reduced sulfate level. In addition, the process would be both environmentally and physiologically safe, i.e. would not involve the use of organic solvents, so that secondary products containing organically bound chlorine would be avoided and the foaming problems and susceptibility to hydrolysis during addition of the alkyl (ether) sulfates would be reduced. In addition, the process would give polyol alkylethers with a high monoalkyl polyol ether content (>80% by weight).

### DESCRIPTION OF THE INVENTION

The present invention relates to a process for the production of polyol alkylethers in which a polyol is deprotonated with a base, preferably alkali metal or alkaline earth metal oxides, carbonates or hydroxides, and

(a) the water formed is continuously removed from the reaction product and the deprotonated polyol is reacted with alkyl and/or alkenyl (ether) sulfates or

(b) sulfuric acid alkylesters are added to the deprotonated polyol after addition of a base, preferably alkali metal or alkaline earth metal oxides, carbonates or hydroxides, and the water formed is continuously removed from the reaction mixture,

the sulfate salt present in the reaction product is precipitated on completion of the reaction by addition of 10 to 20 mol, preferably 11 to 15 mol and more particularly 12 to 13 mol water per mol alkyl (ether) sulfate, alkenyl (ether) sulfate or sulfuric acid alkyl ester at a temperature of 50 to 100° C. and preferably 80 to 90° C. and the polyol alkyl ether obtained is separated from the aqueous and solid phases by methods known per se.

It has surprisingly been found that polyol alkyl ethers can be produced by reaction of polyols with bases and with alkyl (ether) sulfates, alkenyl (ether) sulfates or sulfuric acid alkyl esters and that the sulfate salt present in the reaction mixture can be precipitated during working up by addition of a particular quantity of water and then filtered off. The polyol alkyl ethers thus obtained have a high content of monoalkyl polyolethers. It is particularly advantageous that the unreacted polyols in the aqueous phase can be worked up relatively easily by virtue of the reduced sulfate level and can thus be made accessible to other applications or may be returned as starting component to the process. In addition, solvents do not have to be added to isolate the polyol alkyl ethers. The use of water-free alkyl and/or alkenyl (ether) sulfates, for example in powder or granule form, leads to a reduction in the hydrolysis of the alkyl (ether) sulfates during the reaction. Polyol alkyl ethers can thus be obtained in a particularly effective, inexpensive and environmentally friendly manner.

Polyols

Suitable polyols contain at least two hydroxyl groups. Typical examples are

glycerol,

alkylene glycols such as, for example, ethylene glycol, diethylene glycol, polyethylene glycols with an average molecular weight of 100 to 1,000 dalton, propylene glycol and polypropylene glycol;

technical oligoglycerol mixtures with a degree of self-condensation of 1.5 to 10 such as, for example, technical diglycerol mixtures with a diglycerol content of 40 to 50% by weight and pure diglycerol;

methylol compounds such as, in particular, trimethylol propane;

sugar alcohols containing 4 to 6 carbon atoms, i.e. tetritols, pentitols such as, preferably, threitol, erythritol, ribitol, arabitol, xylitol and, preferably, erythritol, xylitol or mannitol.

Glycerol, alkylene glycols, technical oligoglycerol mixtures, methylol compounds, sugar alcohols and addition products thereof with ethylene and/or propylene oxide are preferably used as polyols for the purposes of the invention, particularly preferred polyols being glycerol, diethylene glycol, diglycerol and other technical oligoglycerol mixtures, trimethylolpropane and xylitol, above all glycerol.

Alkyl and/or Alkenyl (Ether) Sulfates

Alkyl and/or alkenyl (ether) sulfates, which are often also referred to as fatty alcohol (ether) sulfates, are understood to be the sulfation products of primary alcohols which correspond to formula (I):

R¹(A)ₙO—SO₃ (I)

in which R¹ is a linear or branched, aliphatic alkyl and/or alkenyl group containing 6 to 22 carbon atoms and preferably 8 to 18 carbon atoms, A is a C₂H₄O or C₃H₆O group, n is 0 or a number of 1 to 10 and X is an alkali metal and/or alkaline earth metal or ammonium. Typical examples of alkyl (ether) sulfates which may be used in accordance with the invention are the sulfation products of caproic alcohol, caprylic alcohol, capric alcohol, 2-ethyl hexyl alcohol, lauryl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol and erucyl alcohol and the technical mixtures thereof obtained by high-pressure hydrogenation of technical methyl ester fractions or aldehydes from Roelen's oxo synthesis and addition products thereof with 1 to 10 mol ethylene oxide. The sulfation products may advantageously be used in the form of their alkali metal salts and particularly their sodium salts. Alkyl (ether) sulfates based on C_{16/18} tallow fatty alcohols or vegetable fatty alcohols of comparable C chain distribution in the form of their sodium salts and C₈ fatty alcohol, C₁₂ fatty alcohol, C₁₆ fatty alcohol and C₁₈ fatty alcohol are particularly preferred.

In one particular embodiment of the invention, the alkyl and/or alkenyl (ether) sulfate, preferably the sodium alkyl and/or alkenyl (ether) sulfate, may be added in the form of granules or powder, more particularly in the form of granules, preferably in water-free form. Alkyl (ether) sulfate granules are relatively easy to dose and are characterized by reduced dust emission in use. Water-free in the context of the invention means a water content of 0.01 to 5% by weight, preferably 0.1 to 3% by weight and more particularly 0.4 to 2% by weight. The Cognis products Lanette-E, Texapon-K-12-G, Texapon-K-12-P, Sulfopon-1281-G, Texapon-CPS and Texapon-EHS-P are preferably used.

Sulfuric Acid Alkyl Esters

Sulfuric acid alkyl esters can be industrially produced by SO₃ or chlorosulfonic acid (CSA) sulfation of fatty alcohol. Sulfuric acid alkyl esters corresponding to formula (II):

R²(A)ₘSO₃H (II)

in which R² is a linear or branched alkyl and/or alkenyl radical containing 6 to 22 carbon atoms, A is a C₂H₄O or C₃H₆O group and m is 0 or a number of 1 to 10, are suitable for the purposes of the invention. Typical examples are the sulfuric acid esters of caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol and technical mixtures thereof. The sulfuric acid esters may have both a conventional homolog distribution and a narrow homolog distribution. It is particularly preferred to use sulfuric acid alkyl esters based on adducts of technical C_{12/14} or C_{16/18} coconut fatty alcohol fractions and C₈ fatty alcohol, C₁₂ fatty alcohol, C₁₆ fatty alcohol and C₁₈ fatty alcohol.

The use of acidic sulfuric acid alkyl esters in the process according to the invention has the advantage that the neutralization step using aqueous sodium hydroxide and the subsequent removal of water to give the alkyl (ether) sulfates do not have to be carried out at the forefront of the reaction using complicated equipment, instead the acidic sulfuric acid esters may be directly used.

Process

The polyol alkyl ethers according to the invention, during whose production polyol/water mixtures, preferably glycerol/water mixtures, with a low sulfate salt content accumulate, are produced by reaction of polyol anions with alkyl and/or alkenyl (ether) sulfates or sulfuric acid alkyl esters. To this end, the polyol is introduced into the reactor first and alkali metal or alkaline earth metal oxides, carbonates or hydroxides, preferably alkali metal or alkaline earth metal hydroxides and, more particularly, sodium hydroxide, preferably 25 to 50% by weight sodium hydroxide, are slowly added dropwise as base at a temperature of 110 to 130° C. and preferably at a temperature of 120° C. In the deprotonation step, the water formed is continuously removed at 110 to 130° C. and preferably at 120° C. during or after the dropwise addition of the base. In one particular embodiment of the invention, the water formed during the reaction is removed at temperatures of 110 to 130° C. and preferably 120° C., optionally by application of a vacuum of 150 to 10, preferably 130 to 20 and more particularly 120 to 30 mbar. In this embodiment, a vacuum of 150 to 80 mbar and, more particularly, 120 to 100 mbar is preferably applied at the beginning of the removal of water at the above-mentioned temperature and is only increased to 50 to 10 and, more particularly, 30 to 15 mbar and the end of the removal of water. The alkyl and/or alkenyl (ether) sulfates are then added to the polyol (sum of polyol and deprotonated polyol). The reaction with the alkyl and/or alkenyl (ether) sulfates takes place at a temperature of 150 to 220° C. and preferably at a temperature of 170 to 180° C. The reaction mixture is stirred at that temperature for 7 to 10 hours and, more particularly, for 8 to 9 hours. In one particular embodiment of the invention, the alkyl and/or alkenyl (ether) sulfate, preferably the sodium alkyl and/or sodium alkenyl (ether) sulfate, may advantageously be added in the form of granules or powder, more particularly in the form of granules, preferably in water-free form. In another embodiment of the invention, alkali metal or alkaline earth metal oxides, carbonates or hydroxides and preferably alkali metal or alkaline earth metal hydroxides, more particularly sodium hydroxide (50% aqueous solution), and sulfuric acid alkyl esters are added to the deprotonated polyol and the water formed during the deprotonation or neutralization step is then continuously removed from the reaction mixture at 110 to 130° C. and preferably at 130° C., optionally by application of a vacuum as described above. The reaction of the deprotonated polyol with the sulfuric acid alkyl esters is carried out at a temperature of 150 to 220° C. and preferably at a temperature of 170 to 180° C. To this end, the reaction mixture is preferably stirred for 7 to 10 hours and more particularly for 8 to 9 hours at that temperature.

The reaction is monitored by determination of the anionic surfactant content which should be well below 5, preferably 3 and more particularly 1% by weight, based on the active substance content. The alkyl (ether) sulfates, alkenyl (ether) sulfates or sulfuric acid alkyl esters and the polyol are preferably used in a molar ratio of 1:1 to 1:10, preferably 1:2 to 1:8 and more particularly 1:3 to 1:6. The base and the alkyl (ether) sulfates, alkenyl (ether) sulfates or sulfuric acid alkyl esters are used in a molar ratio of 0.9:1 to 1.5:1, preferably 1.1:1 to 1.4:1 and more particularly 1.2:1 to 1.3:1. If the aqueous phase accumulating during the reaction, which contains unreacted polyols, preferably glycerol, is returned to the process as starting polyol, a molar ratio of base to alkyl (ether) sulfates, alkenyl (ether) sulfates or sulfuric acid alkyl esters of 0.9:1 to 1.3:1 is selected. For working up, the reaction mixture is mixed with 10 to 20, preferably 11 to 15 and more particularly 12 to 13 mol water per mol alkyl (ether) sulfate, alkenyl (ether) sulfate or sulfuric acid alkyl esters and optionally with 1 to 20 and preferably 5 to 10 ml of 10 to 70% and preferably 50% base (see above), preferably alkali metal hydroxide, per mol alkyl (ether) sulfate, alkenyl (ether) sulfate or sulfuric acid alkyl ester at a temperature of 50 to 100 and preferably 70 to 90° C. The mixture is then left to undergo phase separation and the phases formed are removed. To this end, either the sulfate salt is first filtered off and the polyol alkyl ether obtained is then removed from the aqueous phase or the polyol alkyl ether obtained is removed from the aqueous phase and the sulfate salt is then filtered off from the aqueous phase. In one particular embodiment of the invention, the aqueous phase accumulating after working up, which contains unreacted polyols, may be filtered in known manner (removal of the sulfate precipitated) and dried and returned as starting polyol to the process according to the invention. The upper organic phase (polyol ether phase) is washed with 100 to 500, preferably 200 to 300 and more particularly 250 ml water per mol alkyl (ether) sulfate, alkenyl (ether) sulfate or sulfuric acid alkyl ester at a temperature of 70 to 95° C. and the phases are again separated.

The organic phase is freed from the water by vacuum distillation and the polyol alkyl ether remains behind as the distillation residue. A preferred reaction product contains—based on the total concentration—ca. 80 to 95 and preferably 90% by weight polyol monoalkyl and 20 to 5 and preferably 10% by weight polyol dialkyl ethers. The sulfate contents of the polyol alkyl ethers are preferably in the range from 0 to 5 and more particularly 0.1 to 2% by weight, based on the active substance content.

Commercial Applications

The polyol alkyl ethers according to the invention may be used in any surface-active preparations known to the expert, preferably in laundry and dishwashing detergents, domestic cleaners and cosmetic and/or pharmaceutical preparations and more particularly in cosmetic hair and body care preparations and in cleaning compositions. These surface active preparations may contain pearlizing waxes, consistency factors, thickeners, superfatting agents, stabilizers, silicone compounds, fats, waxes, lecithins, phospholipids, antioxidants, deodorants, antiperspirants, antidandruff components, swelling agents, tyrosine inhibitors, hydrotropes, solubilizers, preservatives, perfume oils, dyes, other surfactants and the like as further auxiliaries and additives. Suitable cosmetic and/or pharmaceutical preparations are, for example, oral and dental care preparations, hair shampoos, hair lotions, foam baths, shower baths, creams, gels, lotions, alcoholic and aqueous/alcoholic solutions and emulsions.

### EXAMPLES

1. Production of C₁₂ Glycerol Ether by Using Na Alkyl Sulfate Powder

552 g (6 mol) glycerol were heated to 120° C. in a 2-liter four-necked flask, 100 g (1.25 mol) 50% sodium hydroxide were slowly added dropwise and the water formed was continuously removed by condensation at 120° C. under a vacuum of 100 mbar. At the end of the removal of water, the vacuum was reduced to 10 mbar. 300 g (1 mol) Texapon® K12P (sodium lauryl sulfate powder) were added to the Na glycerolate thus formed and suspended therein and, after heating to 180° C., the suspension was stirred at that temperature for 8 hours. The reaction was monitored by determination of the anionic surfactant content which, after 8 hours, was well below 1%. For working up, the reaction mixture was mixed with 225 ml of water and 5 ml of 50% sodium hydroxide at a temperature of 90° C. and the resulting mixture was left standing for phase separation to occur. The phases formed were then separated. To this end, the lower phase was filtered to remove the sodium sulfate precipitated and the upper organic phase (glycerol ether phase) was washed with 250 ml water at a temperature of 90° C. and the phases were again separated. The organic phase was freed from water by vacuum distillation. The C₁₂ glycerol ether remained behind as the distillation residue. The product contained ca. 90% monolauryl glycerol ether and 10% dilauryl glycerol ether.

2. Production of C_{16/18} Glycerol Ether by Using Na Alkyl Sulfate Powder

552 g (6 mol) glycerol were heated to 120° C. in a 2-liter four-necked flask, 100 g (1.25 mol) 50% sodium hydroxide were slowly added dropwise and the water formed was continuously removed by condensation at 120° C. under a vacuum of 100 mbar. At the end of the removal of water, the vacuum was reduced to 10 mbar. 380 g (1 mol) Lanette E powder (sodium cetostearyl sulfate powder) were added to the Na glycerolate thus formed and suspended therein and, after heating to 180° C., the suspension was stirred at that temperature for 8 hours. The reaction was monitored by determination of the anionic surfactant content which, after a reaction time of 8 hours, was well below 1%. For working up, the reaction mixture was mixed with 225 ml of water and 5 ml of 50% sodium hydroxide at a temperature of 90° C. and the resulting mixture was left standing for phase separation to occur. The phases formed were then separated. To this end, the lower phase was filtered to remove the sodium sulfate precipitated and the upper organic phase (glycerol ether phase) was washed with 250 ml water at a temperature of 90° C. and the phases were again separated. The organic phase was freed from water by vacuum distillation. The C_{16/18} glycerol ether remained in the distillation residue. The product contained ca. 70% mono-C_{16/18}-glycerol ether and ca. 16% di-C_{16/18}-glycerol ether.

3. Production of C₁₂ Glycerol Ether by Using Na Alkyl Sulfate Granules

552 g (6 mol) glycerol were heated to 120° C. in a 2-liter four-necked flask, 100 g (1.25 mol) 50% sodium hydroxide were slowly added dropwise and the water formed was continuously removed by condensation at 120° C. under a vacuum of 100 mbar. At the end of the removal of water, the vacuum was reduced to 10 mbar. 300 g (1 mol) Texapon® K12G (sodium lauryl sulfate granules) were added to the Na glycerolate thus formed and suspended therein and, after heating to 180° C., the suspension was stirred at that temperature for 8 hours. The reaction was monitored by determination of the anionic surfactant content which, after 8 hours, was well below 1%. For working up, the reaction mixture was mixed with 225 ml of water and 5 ml of 50% sodium hydroxide at a temperature of 90° C. and the resulting mixture was left standing for phase separation to occur. The phases formed were then separated. To this end, the lower phase was filtered to remove the sodium sulfate precipitated and the upper organic phase (glycerol ether phase) was washed with 250 ml water at a temperature of 90° C. and the phases were again separated. The organic phase was freed from water by vacuum distillation. The C₁₂ glycerol ether remained behind as the distillation residue. The product contained ca. 90% monolauryl glycerol ether and 10% dilauryl glycerol ether.

4. Production of C₁₂ Glycerol Ether by Neutralization with Acidic Na Alkyl Sulfuric Acid Esters

In a 2-liter four-necked flask, 213 g (0.8 mol) C₁₂ sulfuric acid ester (prepared from C₁₂ fatty alcohol by sulfation with SO₃) were added dropwise to a mixture of 400 g (4.37 mol) Na glycerolate (prepared from 4.37 mol glycerol and 0.87 mol 50% sodium hydroxide) and 64 g (0.8 mol) 50% sodium hydroxide. The water present was removed in vacuo at 100° C. The reaction mixture was then stirred for 8 hours at 180° C. The reaction was monitored by determination of the anionic surfactant content which, after 8 hours, was below 3%. For working up, the reaction mixture was mixed with 225 ml of water and 5 ml of 50% sodium hydroxide at a temperature of 90° C. and the resulting mixture was left standing for phase separation to occur. The phases formed were then separated. To this end, the lower phase was filtered to remove the sodium sulfate precipitated and the upper organic phase (glycerol ether phase) was washed with 250 ml water at a temperature of 90° C. and the phases were again separated. The organic phase was freed from water by vacuum distillation. The C₁₂ glycerol ether remained behind as the distillation residue. The product contained ca. 80% monolauryl glycerol ether and 8% dilauryl glycerol ether.

5. Production of C₁₂ Glycerol Ether by Re-Using the Polyol/Water Mixture Removed in Example 3 and Reacting it with Na Alkyl Sulfate Granules

In a 2-liter four-necked flask, 527 g polyol/water mixture (phase removed in Example 3) were freed from the water at 120° C. under a vacuum of 100 mbar, leaving 380 g (4.1 mol) glycerol which was supplemented with an additional quantity of 172 g (1.9 mol) glycerol. This corresponded to a total quantity of 552 g (6 mol) glycerol. The glycerol was slowly heated to 120° C., 80 g (1 mol) 50% sodium hydroxide were slowly added dropwise and the water formed was continuously removed by condensation at 120° C. under a vacuum of 100 mbar. At the end of the removal of water, the vacuum was reduced to 10 mbar. 300 g (1 mol) Texapon® K12G (sodium lauryl sulfate granules) were added to the Na glycerolate thus formed and suspended therein and, after heating to 180° C., the suspension was stirred at that temperature for 8 hours. The reaction was monitored by determination of the anionic surfactant content which, after 8 hours, was well below 1%. For working up, the reaction mixture was mixed with 225 ml of water and 5 ml of 50% sodium hydroxide at a temperature of 90° C. and the resulting mixture was left standing for phase separation to occur. The phases formed were then separated. To this end, the lower phase was filtered to remove the sodium sulfate precipitated and the upper organic phase (glycerol ether phase) was washed with 250 ml water at a temperature of 90° C. and the phases were again separated. The organic phase was freed from water by vacuum distillation. The C₁₂ glycerol ether remained behind as the distillation residue. The product contained ca. 80% monolauryl glycerol ether and ca. 10% dilauryl glycerol ether.

1-11. (cancelled).

12. A process for making polyol alkyl ether comprising:
(a) providing a polyol;
(b) deprotonating the polyol with a base to form a first reaction product;
(c) continuously removing water from the first reaction product to form a second reaction product;
(d) providing an alk(en)yl (ether) sulfate;
(e) reacting the alk(en)yl (ether) sulfate with the second reaction product to form a third reaction product containing a sulfate salt;
(f) precipitating the sulfate salt from the third reaction product by adding from about 10 to 20 mol of water per mol of alk(en)yl (ether) sulfate to the third reaction product, at a temperature of from about 50 to 100° C.;
(g) forming an aqueous and/or solid phase containing the polyol alkyl ether; and
(h) separating the polyol alkyl ether from the aqueous and/or solid phase.

13. The process of claim 12 wherein the polyol of step (a) is glycerol.

14. The process of claim 12 wherein (c) is performed at a temperature of from about 110 to 130° C.

15. The process of claim 12 wherein (e) is performed at a temperature of from about 150 to 220° C.

16. The process of claim 12 wherein the alk(en)yl (ether) sulfate of step (d) and the polyol of step (a) are employed in a molar ratio of from 1:1 to 1:10.

17. The process of claim 12 wherein (f) is performed by adding from about 11 to 15 mol of water per mol of alk(en)yl (ether) sulfate, at a temperature of from about 50 to 100° C.

18. The process of claim 12 further comprising removing unreacted polyol from the aqueous phase of step (g) or (h), and then re-using it in step (a).

19. The process of claim 12 wherein the alk(en)yl (ether) sulfate of step (d) has a water content of from about 0.1 to 5% by weight, based on the weight of the alk(en)yl (ether) sulfate.

20. The process of claim 12 wherein the alk(en)yl (ether) sulfate of step (d) has a water content of from about 0.1 to 3% by weight, based on the weight of the alk(en)yl (ether) sulfate.

21. The process of claim 12 wherein the base of step (b) and the alk(en)yl (ether) sulfate of step (d) are employed in a molar ratio of from 0.9:1 to 1.5:1.

22. A process for making polyol alkyl ether comprising:
(a) providing a polyol;
(b) deprotonating the polyol with a base to form a first reaction product;
(c) providing a sulfuric acid alkylester;
(d) adding the sulfuric acid alkyl ester to the first reaction product to form a second reaction product;
(e) continuously removing water from the second reaction product to form a third reaction product containing a sulfate salt;
(f) precipitating the sulfate salt from the third reaction product by adding from about 10 to 20 mol of water per mol of alk(en)yl (ether) sulfate to the third reaction product, at a temperature of from about 50 to 100° C.;
(g) forming an aqueous and/or solid phase containing the polyol alkyl ether; and
(h) separating the polyol alkyl ether from the aqueous and/or solid phase.

23. The process of claim 22 wherein the polyol of step (a) is glycerol.

24. The process of claim 22 wherein (e) is performed at a temperature of from about 110 to 130° C.

25. The process of claim 22 wherein (d) is performed at a temperature of from about 150 to 220° C.

26. The process of claim 22 wherein the sulfuric acid alkylester of step (c) and the polyol of step (a) are employed in a molar ratio of from 1:1 to 1:10.

27. The process of claim 22 wherein the base of step (b) and the sulfuric acid alkylester of step (c) are employed in a molar ratio of from 0.9:1 to 1.5:1.

28. The process of claim 22 wherein (f) is performed by adding from about 11 to 15 mol of water per mol of alk(en)yl (ether) sulfate, at a temperature of from about 50 to 100° C.

29. The process of claim 22 further comprising removing unreacted polyol from the aqueous phase of step (g) or (h), and then re-using it in step (a).
